(19) United States
(12) Patent Application Publication
(10) Pub. No.: US 2023/0123525 A1
(43) Pub. Date: Apr. 20, 2023

(54) SURGICAL INSTRUMENT WITH A LOOP MADE OF A METAL WIRE

(71) Applicant: RZ-Medizintechnik GmbH, Tuttlingen (DE)

(72) Inventors: Alexander DOPPELSTEIN, Spaichingen (DE); Tobias ZUBROD, Tuttlingen (DE)

(21) Appl. No.: 17/966,223

(22) Filed: Oct. 14, 2022

(30) Foreign Application Priority Data
Oct. 15, 2021 (DE) 20 2021 105 657.6

(51) Int. Cl. A61B 18/14

(52) U.S. Cl. CPC A61B 18/14; A61B 2018/141; A61B 2018/126

(57) ABSTRACT

A surgical instrument with a shaft is constructed as a hollow tube and has a longitudinal axis, proximal and distal ends and a metal wire having a first end section, a shaft section and a working section. The first end section is arranged in the area of the proximal end of the shaft or is guided out of the shaft on the proximal end. The shaft section is guided in the hollow tube and the wire is arranged such that it can move along the longitudinal axis of the shaft into a first position of the working section on the distal end projecting out of the shaft and into a second position of the working section within the shaft. The instrument is constructed as a bipolar instrument, wherein the metal wire is part of a first electrode and a second electrode is arranged in some sections on the shaft.

### CROSS-REFERENCE TO RELATED APPLICATIONS

This application claims priority under 35 U.S.C. § 119(b) to German Patent Application No. 20 2021 105 657.6, filed on Oct. 15, 2021, the disclosure of which is incorporated herein by reference in its entirety.

### BACKGROUND OF THE INVENTION

The invention relates to a surgical instrument with a loop made of a metal wire.

Surgical instruments with a shaft that has a longitudinal axis, a proximal end, and a distal end, and with a loop made of a metal wire are known.

The metal wire can carry electrical power, but it can also be used without power. If the metal wire is used without power, the surgical instrument can be used, for example, for dilation or dissection of tissue. If the metal wire carries electrical power, the surgical instrument can be used for the resection of tissue, for example, polyps or fibroids, for the coagulation of tissue, or for the vaporization of tissue. Here, the instrument can be used both in a monopolar design and also in a bipolar design.

DE 10 2017 126 199 A1 discloses a surgical instrument with a shaft constructed as a hollow tube and with a loop made of a metal wire, wherein the metal wire projects out of the shaft with a loop section on the distal end, wherein means are provided, with which the size of the loop can be varied. For this purpose, the wire can be moved in the hollow tube along the longitudinal axis of the shaft, wherein the size of the loop becomes larger the larger the section of the wire projecting out of the shaft on the distal end of the shaft becomes. The loop section is thus guided on the distal end of the hollow tube into two tubular insulation elements, by means of which, in addition to the size, the angle of the loop can also be varied.

### BRIEF SUMMARY OF THE INVENTION

The task of the invention is to further develop a surgical instrument with a working section made of a metal wire such that it can be used in minimally invasive surgery.

The task of the invention is realized by a surgical instrument with the preferred features.

Advantageous constructions and refinements of the invention are specified in the dependent claims.

The surgical instrument according to the invention with a shaft that is constructed as a hollow tube and that has a longitudinal axis, a proximal end, and a distal end, and with a metal wire that has a first end section, at least one shaft section, and a working section, wherein the first end section is arranged in the area of the proximal end of the shaft or is guided out of the shaft on the proximal end of the shaft, wherein the shaft section is guided in the hollow tube and wherein the wire is arranged such that it can move along the longitudinal axis of the shaft into a first position of the working section on the distal end projecting out of the shaft and into a second position of the working section completely within the shaft, is constructed as a bipolar instrument, wherein the metal wire is part of a first electrode and a second electrode is arranged at least in some sections on the shaft. The arrangement of the working section completely within the shaft has the result that the distal end has the fewest possible projections, hooks, or edges, and thus the distal end can also be used in minimally invasive surgeries, without the risk of injuries due to a protruding wire section.

In an especially preferred way, at least the working section, preferably the metal wire, is made from a shape memory alloy, for example, from nitinol. This allows the desired shape of the working section to be achieved in a simple way, for example, the shape of an angled hook or a loop, when the working section is pushed out from the shaft on the distal end.

Preferably, the metal wire has the first end section, two shaft sections, wherein the working section is arranged between the two shaft sections, and a second end section. This configuration enables a stable positioning of the working section, especially when it forms a loop.

In an especially preferred way, the working section forms a loop to be able to remove tissue during an operation in a simple way.

In the first position, the loop advantageously has a diameter of up to 10 mm. This allows flexible use of the surgical instrument.

Advantageously, the shaft has, starting from the distal end, a distal end section, in which a first section of the second electrode is arranged on an outer wall of the shaft or forms this end section, wherein a second section of the second electrode extending from the distal end section to the proximal end of the shaft is arranged on an inner wall of the shaft. Such a configuration allows the most compact construction of the instrument as a bipolar instrument.

According to one advantageous refinement of the invention, exactly one tubular insulation element is arranged in the shaft on the distal end of the shaft. The metal wire is here guided in particular through the tubular insulation element both from the inside of the shaft at the distal end to the outside and also in particular through the same opening from the outside back into the inside of the shaft, which means that the wire and in particular the working section can be retracted completely into the inside of the shaft.

One preferred refinement of the invention provides that the two shaft sections are arranged in the hollow tube such that they are insulated from one another so as to be able to avoid a short circuit.

According to one preferred embodiment of the invention, the shaft has an outer diameter in the range from 1.9 mm to 2.5 mm, preferably in the range from 2.0 mm to 2.2 mm. Such dimensioning allows the surgical instrument to be used in minimally invasive surgery.

Preferably, the metal wire has a diameter of less than 1 mm, preferably a diameter in the range from 0.3 mm to 0.7 mm. A wire with such a construction is especially suitable for minimally invasive applications.

Advantageously, the shaft has an axial length of approximately 350 mm to 450 mm, preferably of approximately 390 mm to 410 mm, especially preferred of approximately 401 mm. Such a length also allows access to locations in the body that are not directly under the skin, even in the case of a minimally invasive procedure.

According to one preferred embodiment of the invention, the shaft is designed so that it can bend for the surgical instrument to be better able to adapt to the specific anatomical conditions during different operations.

One advantageous construction of the invention provides that the distal end section has an axial length of approximately 10 mm to 20 mm, preferably of approximately 15 mm to 17 mm, especially preferred of approximately 18 mm. As a result, a second electrode can be provided with a sufficiently large surface area.

According to one advantageous construction of the invention, the instrument has an adjustment element, to which at least the first end section is attached, wherein the adjustment element is mounted so that either it can move in the direction of the longitudinal axis of the shaft or it can rotate about an axis. If present, a second end section can also be attached to the adjustment element. With the help of such an adjustment element, it is possible to shorten or lengthen the length of the section of the metal wire protruding out from the shaft at the distal end and, in this way, to vary the size of the loop, for example, in the case that the working section forms a loop.

In an especially preferred way, a grip element, in which the adjustment element is arranged, is arranged at the proximal end of the shaft, which makes it possible to provide simple operation of the surgical instrument.

According to one preferred embodiment of the invention, the shaft is supported against the grip element so that it can rotate about its longitudinal axis. This allows the plane of the loop to rotate about the longitudinal axis of the shaft, without the grip element having to rotate, which enables flexible use during an operation.

### BRIEF DESCRIPTION OF THE SEVERAL VIEWS OF THE DRAWING

The foregoing summary, as well as the following detailed description of the preferred invention, will be better understood when read in conjunction with the appended drawings. For the purpose of illustrating the preferred invention, there are shown in the drawings embodiments which are presently preferred. It should be understood, however, that the invention is not limited to the precise arrangements and instrumentalities shown. In the drawings:

FIG. 1 is a top perspective view of a first embodiment of a surgical instrument according to the preferred invention with a working section constructed as a loop in a first position,

FIG. 2 is a top perspective view of the surgical instrument according to FIG. 1 with the working section partially retracted into the shaft,

FIG. 3 is a top perspective view of the surgical instrument according to FIG. 1 with the working section completely retracted into the shaft,

FIG. 4 is a side elevational view of the surgical instrument according to FIG. 1,

FIG. 5 is a top plan view of the surgical instrument according to FIG. 1,

FIG. 6 is an enlarged detail of a front elevational view of the surgical instrument according to FIG. 1,

FIG. 7 is a side elevational view of the shaft with the wire of the surgical instrument according to FIG. 1 arranged therein,

FIG. 8 is a front elevational view of the shaft according to FIG. 7,

FIG. 9 is a longitudinal cross-section of the shaft according to FIG. 7, taken along line A-A of FIG. 7,

FIG. 10 is an enlarged detail cross-sectional view of the distal end of the shaft according to FIG. 9, taken from within the circle of FIG. 9,

FIG. 11 is a top perspective view of a second embodiment of a surgical instrument according to the preferred invention with a working section constructed as a loop in a first position, and

FIG. 12 is a top perspective view of a third embodiment of a surgical instrument according to the preferred invention with a working section constructed as a blade in a first position.

### DETAILED DESCRIPTION OF THE INVENTION

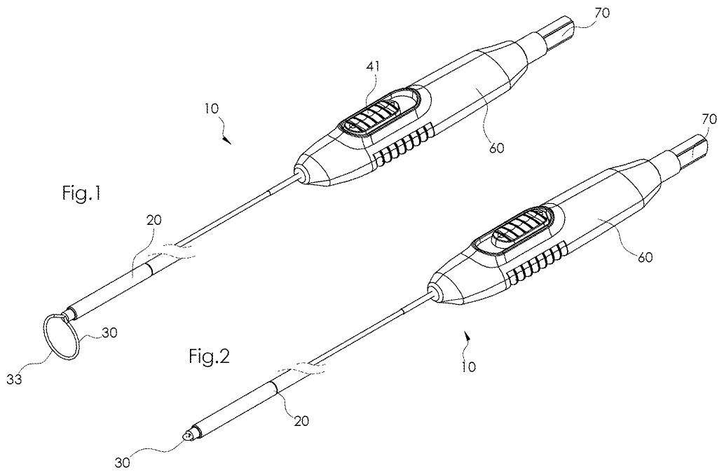

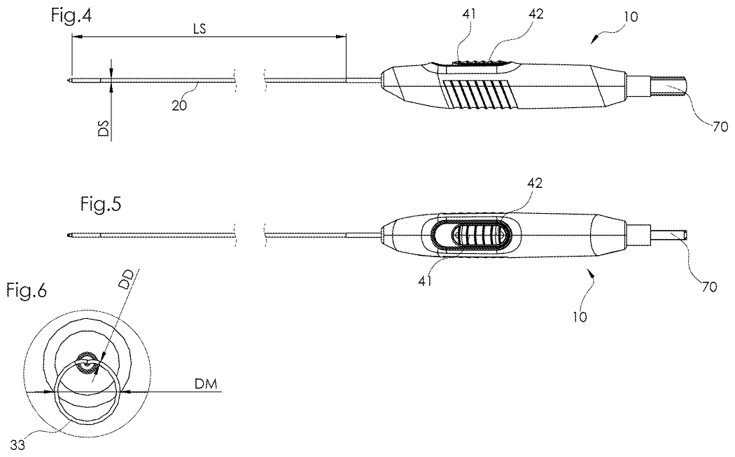

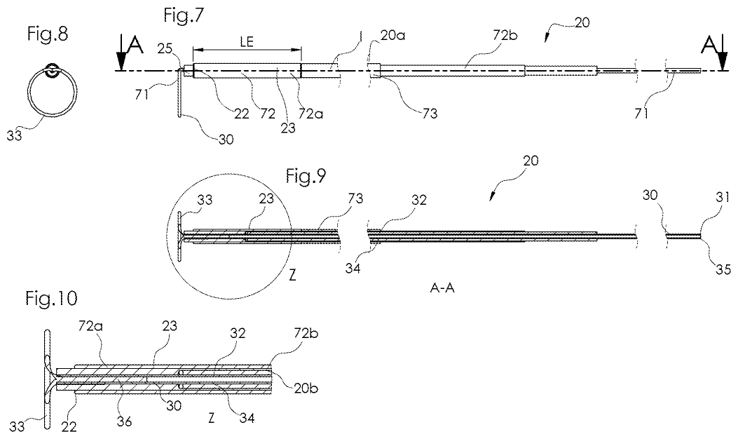

FIGS. 1 to 10 show different views of a first embodiment of a surgical instrument 10, wherein not all the reference symbols have been indicated in all the figures for the sake of better clarity.

The surgical instrument 10 has a shaft 20 that has a longitudinal axis l, a proximal end 21, and a distal end 22, and a metal wire 30.

The shaft 20 is constructed as a hollow tube. Furthermore, the shaft 20 has an outer wall 20a and an inner wall 20b. The shaft 20 can be constructed, in particular, as a metal tube. The shaft 20 can have an outer diameter DS in the range from 1.9 mm to 2.5 mm, preferably in the range from 2.0 mm to 2.2 mm. Furthermore, the shaft 20 can have an axial length LS of approximately 350 mm to 450 mm, preferably of approximately 390 mm to 410 mm, especially preferred of approximately 401 mm.

A grip element 60 can be arranged at the proximal end 21 of the shaft 20, especially such that the shaft 20 is mounted against the grip element 60 so that it can rotate about its longitudinal axis l. The grip element 60 can have a housing that can be assembled, in particular, from two housing shells.

In this embodiment, the metal wire 30 has a first end section 31, a first shaft section 32, a working section 33, a second shaft section 34, and a second end section 35. The first end section 31 can be guided out from the shaft 20 in the area of the proximal end 21 of the shaft 20 and can be arranged, in particular, in the grip element 60. The first shaft section 32 is arranged in the shaft 20 and runs in particular from the proximal end 21 to the distal end 22 of the shaft 20. The working section 33 can be guided out of the shaft 20 in a first position of the metal wire 30 at the distal end 22 of the shaft 20 and can form a loop. The metal wire 30 is then guided through the distal end 22 back into the shaft 20, in particular, such that the second shaft section 34 is arranged in the shaft 20 and runs essentially parallel to the first shaft section 32. The second end section 35 of the metal wire 30 can also be guided out of the proximal end 21 of the shaft 20. Alternatively, the second end section 35 can be fixed in the shaft 20.

The metal wire 30 has, in particular, a one-piece design. The metal wire 30 can have a diameter DD of less than 1 mm, preferably a diameter DD in the range from 0.3 mm to 0.7 mm (see FIG. 5).

At least the working section 33, but preferably the complete metal wire 30, can be made from a shape memory alloy, for example, nitinol.

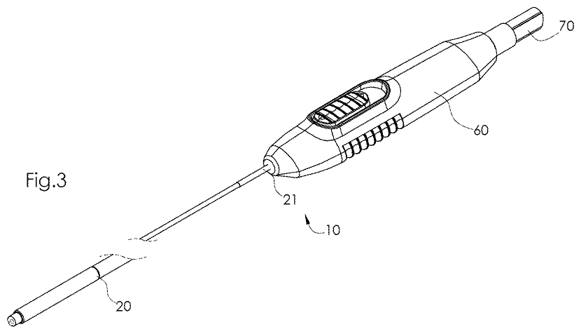

The metal wire 30 is arranged so that it can move along the longitudinal axis l of the shaft 20, in particular, between a first position and a second position. In the first position of the metal wire 30, the working section 33 can be guided out of the shaft 20 at the distal end 22 of the shaft 20 and can form a loop (see, in particular, FIG. 1). In the second position, the metal wire 30 is pulled into the shaft 20 so that the working section 33 is arranged completely within the shaft 20 (see FIG. 3). Between the two positions, a shorter section of the working section 33 if guided out of the distal end 22 of the shaft 20, so that a loop can be formed with a smaller diameter than in the first position (see FIG. 2). In the first position, the loop can have a diameter DM of up to 10 mm, preferably of up to 30 mm (see FIG. 5).

For moving the wire 30, the surgical instrument 10 can have a first adjustment element 41, to which at least the first end section 31 of the metal wire 30 is attached, for example, it is fixed by clamping. The second end section 35 can also be arranged on the adjustment element 41. Here, the first adjustment element 41 is arranged so that it can move in the direction of the longitudinal axis l of the shaft 20 (see FIGS. 1 and 2).

The first adjustment element 41 can have an actuation element 42 that can be guided through a groove arranged in the housing of the grip element 60 on the outside of the housing of the grip element 60, wherein the actuation element 42 is arranged so that it can move in the groove. If the first adjustment element 41 is pushed in the direction of the longitudinal axis l, for example, in the direction toward the shaft 20, the first shaft section 32 in the shaft 20 is pushed in the direction toward the distal end 22 of the shaft 20 and the length of the working section 33 that protrudes out of the shaft 20 at the distal end 22 increases, so that a larger loop can be formed. If the first adjustment element 41 moves in the direction parallel 20 to the longitudinal axis l away from the shaft 20, the shaft section 32 is pulled out from the shaft 20 at the proximal end 21 and in this way, the length of the working section 33 protruding out of the shaft 20 at the distal end 22 is shortened, so that a smaller loop is formed (see, in particular, FIG. 2). Different positions of the actuation element 42 that correspond to a certain size of the loop formed by the working section 33 can be marked on the groove. If the actuation element 42 is finally completely retracted or, in other words, moved to the position farthest away from the shaft 20, the wire 30 is arranged completely within the shaft 20 (see FIG. 3).

The surgical instrument 10 is constructed as a bipolar instrument with a first electrode 71 and a second electrode 72. The metal wire 30 is part of the first electrode 71. The second electrode 72 is arranged at least in some sections on the shaft 20. For this purpose, the shaft 20 can have, starting from the distal end 22, a distal end section 23, in which a first section 72a of the second electrode 72 is arranged on the outer wall 20a of the shaft 20 or forms this end section 23 of the shaft 20, while a second section 72b of the second electrode 72 extending from the distal end section 23 to the proximal end 21 of the shaft 20 is arranged on the inner wall 20b of the shaft 20. An electrically insulating layer can be arranged on the second section 72b of the second electrode 72. The distal end section 23 can have an axial length LE of approximately 10 mm to 20 mm, preferably of approximately 15 mm to 17 mm, especially preferred of approximately 18 mm.

On the distal end 22 of the shaft 20, exactly one tubular insulation element 25 is arranged in the shaft 20. Through this, the metal wire 30 is guided out from the interior of the shaft 20 and back into the shaft 20 on the distal end 22. In the area of the distal end 22, the insulation element 25 insulates the first electrode 71, i.e., in particular, the metal wire 30, against the second electrode 72. The metal wire 30 is arranged against the tubular insulation element 25 so that it can move in the axial direction, while the tubular insulation element 25 is arranged axially fixed in the shaft 20. The metal wire 30 can be pushed, in particular, with the working section 33, completely into the shaft 20, in particular, the tubular insulation element 25. The two shaft sections 32, 34 of the metal wire 30 can be arranged insulated from one another in the shaft 20, for example, in the insulation element 25. For example, in this way, the two shaft sections 32, 34 can be fixed in position relative to each other in some sections by means of an insulation element 36 such that they are both fixed on the insulation element 36 without touching each other (see FIG. 9).

The surgical instrument 10 can have a power connection 70, for example, in the form of a connector for connecting to a corresponding cable with a connector arranged thereon. Alternatively, there is also the possibility that the surgical instrument 10 has a cordless power supply, for example, with the help of a battery or rechargeable battery.

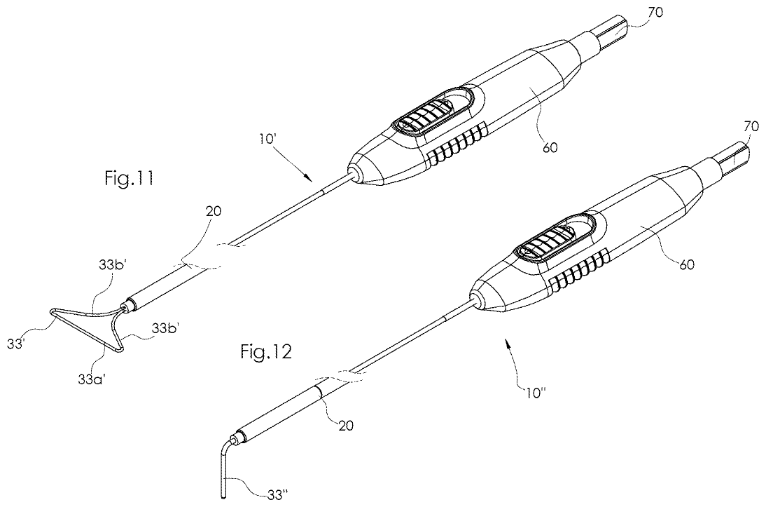

FIG. 11 shows a perspective view of a second embodiment of a surgical instrument 10′ according to the invention, which differs from the surgical instrument 10 shown in FIGS. 1 to 10 only in the shape of the loop. While the working section 33 forms an essentially circular loop in the first embodiment of the instrument 10, the surgical instrument 10′ of the second embodiment has a working section 33′ that forms a loop that comprises a linear section 33a′ arranged between two arc-shaped sections 33b′. Such a linear section 33a′ enables linear processing of tissue.

FIG. 12 shows a perspective view of a third embodiment of a surgical instrument 10″ according to the invention, which differs from the surface instrument 10 shown in FIGS. 1 to 10 in the design of the metal wire 30 and the working section 33. In this embodiment, the metal wire 30 has the first end section 31, the first shaft section 32, and the working section 33″, but not the second shaft section 34 and the second end section 35. The working section 33″ thus forms a free end. In this embodiment, the wire 30 can also be moved between the first position, in which the working section 33″ protrudes at the distal end 22 of the shaft 20 (see FIG. 12) and the second position, in which the working section 33″ is arranged completely within the shaft 20. If the metal wire 30 or at least the working section 33″ is made from a shape memory alloy, this can make it possible for the working section 33″ to move to a desired angle relative to the longitudinal axis l of the shaft 20 in the first position, for example, approximately into a right angle (see FIG. 12). Such a working section 33″ with a free end can be used, for example, like a blade by applying power.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### LIST OF REFERENCE SYMBOLS

10 Surgical instrument
10′ Surgical instrument
10″ Surgical instrument
20 Shaft
20a Outer wall
20b Inner wall
21 Proximal end
22 Distal end
23 Distal end section
25 Insulation element
30 Metal wire
31 First end section
32 First shaft section
33 Working section
33′ Working section
33a′ Section
33b′ Section
33″ Working section
34 Second shaft section
35 Second end section
36 Insulation element
41 Adjustment element
42 Actuation element
60 Grip element
70 Power connection
71 First electrode
72 Second electrode
72a First section
72b Second section
73 Insulating layer
l Longitudinal axis
LS Length
LE Length
DS Outer diameter
DD Diameter
DM Diameter

1. A Surgical instrument comprising:
a shaft that is constructed as a hollow tube and that has a longitudinal axis, a proximal end, and a distal end;
a metal wire that has a first end section, a shaft section, and a working section, wherein the first end section is arranged in the area of the proximal end of the shaft or is guided out of the shaft on the proximal end of the shaft, wherein the shaft section is guided in the hollow tube and wherein the wire is arranged such that the wire can move along the longitudinal axis of the shaft into a first position of the working section on the distal end projecting out of the shaft and into a second position of the working section completely within the shaft, wherein the instrument is comprised of a bipolar instrument, wherein the metal wire is part of a first electrode and a second electrode is arranged at least in some sections on the shaft.

2. The surgical instrument according to claim 1, characterized in that the metal wire is manufactured from a shape memory alloy.

3. The surgical instrument according to claim 1, characterized in that the metal wire has the first end section, and two shaft sections, wherein the working section is arranged between the two shaft sections, and a second end section.

4. The surgical instrument according to claim 1, characterized in that the working section forms a loop.

5. The surgical instrument according to claim 4, characterized in that, in the first position, the loop has a diameter of up to ten millimeters (10 mm).

6. The surgical instrument according to claim 1, characterized in that the shaft has, starting from the distal end, a distal end section, in which a first section of the second electrode is arranged on an outer wall of the shaft or forms the distal end section, and a second section of the second electrode is arranged from the distal end section up to the proximal end of the shaft on an inner wall of the shaft.

7. The surgical instrument according to claim 1, characterized in that, on the distal end of the shaft a tubular insulation element is arranged on the shaft.

8. The surgical instrument according to claim 1, characterized in that the shaft has an outer diameter in a range from one and nine tenths millimeters (1.9 mm) to two and one-half millimeters (2.5 mm).

9. The surgical instrument according to claim 1, characterized in that the metal wire has a diameter of less than one millimeter (1 mm), preferably a diameter (DD) in the range from 0.3 mm to 0.7 mm.

10. The surgical instrument according to claim 1, characterized in that the shaft has an axial length of approximately three hundred fifty millimeters (350 mm) to four hundred fifty millimeters (450 mm).

11. The surgical instrument according to claim 1, characterized in that the distal end section has an axial length of approximately thirty millimeters (30 mm) to twenty millimeters (20 mm).

12. The surgical instrument according to claim 1, characterized in that the instrument has an adjustment element to which at least the first end section is fastened, wherein the adjustment element is mounted so that the adjustment element can either move in a direction of the longitudinal axis of the shaft or can rotate about an axis.

13. The surgical instrument according to claim 12, characterized in that, on the proximal end of the shaft, a grip element is arranged, in which the adjustment element is arranged.

14. The surgical instrument according to claim 2, wherein the shape memory alloy is comprised of nitinol.

15. The surgical instrument according to claim 4, wherein the loop has a diameter of up to thirty millimeters (30 mm).

16. The surgical instrument according to claim 8, wherein the range is from two millimeters (2.0 mm) to two and two tenths millimeters (2.2 mm).

17. The surgical instrument according to claim 9, wherein the diameter is in a range from three tenths millimeters (0.3 mm) to seven tenths millimeters (0.7 mm).

18. The surgical instrument according to claim 10, wherein the axial length of the shaft is approximately three hundred ninety millimeters (390 mm) to four hundred ten millimeters (410 mm).

19. The surgical instrument according to claim 18, wherein the axial length of the shaft is approximately four hundred one millimeters (401 mm).

20. The surgical instrument according to claim 11, wherein the axial length of the distal end section is approximately fifteen millimeters (15 mm) to seventeen millimeters (17 mm).

21. The surgical instrument according to claim 20, wherein the axial length of the distal end section is approximately eighteen millimeters (18 mm).
